(19) United States
(12) Patent Application Publication
(10) Pub. No.: US 2020/0060986 A1
(43) Pub. Date: Feb. 27, 2020

(54) TRANSDERMAL THERAPEUTIC SYSTEM

(71) Applicant: Shin-Etsu Polymer Co., Ltd., Tokyo (JP)

(72) Inventors: Takashi Gonda, Saitama (JP); Naoyuki Yaguchi, Saitama (JP)

(73) Assignee: Shin-Etsu Polymer Co., Ltd., Tokyo (JP)

(21) Appl. No.: 16/552,840

(22) Filed: Aug. 27, 2019

(30) Foreign Application Priority Data
Aug. 27, 2018 (JP) 2018-158051

(51) Int. Cl. A61K 9/70

(52) U.S. Cl. CPC A61K 9/7084

(57) ABSTRACT

A transdermal therapeutic system including: a support layer of a flexible tape; an adhesive layer that is permeable and laminated in a surface of the support layer; a drug that is transdermally absorbable and stored in each of a plurality of storage cavities in the surface of at least the support layer among the support layer and the adhesive layer; and a liner layer detachably laminated in an adhesive surface of the adhesive layer, wherein when a polyether ether ketone resin film having low irritancy is used as the support layer, the polyether ether ketone resin film is used as a sample, and an intradermal reaction test is carried out by using an extract extracted under predetermined conditions in a physiological saline or sesame oil, a difference in average scores of an extract administration site and a blank extract administration site is 1.0 or less in any of extraction media.

This nonprovisional application claims priority under 35 U.S.C. § 119 (a) on Patent Application No. 2018-158051 filed in Japan on 27 Aug. 2018, the entire contents of which are hereby incorporated by reference.

### BACKGROUND OF THE INVENTION

### Field of the Invention

The present invention relates to a transdermal therapeutic system for use in close contact with skin.

A transdermal therapeutic system (TTS) is a pharmaceutical preparation for the purpose of causing an active ingredient to be absorbed into blood from skin and then delivered to an entire body. This transdermal therapeutic system can maintain a sustained concentration of a drug in blood, can prevent the issue of a person forgetting to take a medicine that is to be taken internally, and can be discontinued at any time by detaching the therapeutic system when a side effect occurs, and thus is utilized as a useful therapeutic system.

### Description of the Conventional Art

With reference to Japanese Patent No. 4604238, Japanese Patent Application Laid-Open No. 2017-066123, Japanese Patent Application Laid-Open No. 2007-045917, Japanese Patent Application Laid-Open No. 2006-225522, and Japanese Patent Application Laid-Open No. 2003-063955, while not illustrated, a conventional transdermal therapeutic system is formed in a three-layer structure including a support layer that is flexible, a patch layer laminated in the support layer and used on skin, and a liner layer that is detachably laminated in the patch layer. The support layer is formed from nonwoven fabric, a polyethylene terephthalate resin film or the like. Furthermore, the patch layer is laminated and formed by coating a surface of the support layer with a blended composition of an adhesive and a drug and drying, and an exposed adhesive surface is covered with the liner layer. The liner layer is formed from a polyethylene terephthalate resin film or the like, and covers and protects the adhesive surface that is a surface of the patch layer.

The conventional transdermal therapeutic system is manufactured as described above, and when the support layer is made from nonwoven fabric, the conventional transdermal therapeutic system excels in air permeability and flexibility, but has a significant problem in processability. Furthermore, when the support layer is formed from a polyethylene terephthalate resin film, the conventional transdermal therapeutic system excels in mechanical properties, heat resistance, transparency, and electrical insulating properties, but has problems in hydrolysis resistance, chemical resistance, alkaline resistance, and acid resistance, and there is a concern that improvement in problems cannot be achieved. There is also a concern that the hydrolysis resistance, chemical resistance, alkaline resistance, and acid resistance may become significant problems depending on the type of drug. Further, since a transdermal therapeutic system is used in close contact with skin, it is important that the patch layer and also the support layer have low irritancy to skin.

### BRIEF SUMMARY OF THE INVENTION

In light of the foregoing, an object of the present invention is to provide a transdermal therapeutic system that can improve processability, hydrolysis resistance, chemical resistance, alkaline resistance, acid resistance, and low irritancy of a support layer.

To solve the above problems, a first embodiment of the present invention is a therapeutic system in which an adhesive layer is laminated in a support layer, and a transdermally absorbable drug is provided in at least the support layer among the support layer and the adhesive layer, wherein the support layer is a polyether ether ketone resin film.

In a second embodiment, when the polyether ether ketone resin film is used as a sample, and an intradermal reaction test is carried out by using an extract extracted under predetermined conditions in a physiological saline or sesame oil, a difference in average scores of an extract administration site and a blank extract administration site is 1.0 or less in any of extraction media.

In a third embodiment, a coating protective layer that is detachably laminated in the adhesive layer can be provided.

In a fourth embodiment, a plurality of storage cavities for a drug can be formed by a nanoimprinting method in a laminated surface that is laminated in the adhesive layer of the polyether ether ketone resin film.

In a fifth embodiment, the intradermal reaction test of the polyether ether ketone resin film is preferably carried out by using an extract extracted in a physiological saline or sesame oil at 121° C. for 1 hour in accordance with “Guidance for Biological Safety Test of Medical Device”, an appendix of Notification No. 20 issued by the Director of Office of Medical Devices Evaluation, Evaluation and Licensing Division, Pharmaceutical and Food Safety Bureau, Pharmaceutical and Medical Safety Bureau, Ministry of Health and Welfare on Mar. 1, 2012 and ISO 10993-10:2010.

In a sixth embodiment, a crystallization degree of the polyether ether ketone resin film is less than 20%.

Here, the adhesive layer and the drug in the embodiments may be integrated or separate. When the adhesive layer and the drug are separate, the adhesive layer can include a permeable resin film that is laminated in one surface of the support layer and covers the drug, and an adhesive laminated on the resin film. Furthermore, the adhesive layer can also be formed from only an adhesive that is laminated in one surface of the support layer and covers the drug.

The transdermal therapeutic system according to the present invention can be manufactured by forming recessed portions for storage cavities in a polyether ether ketone resin film, laminating an adhesive layer in one surface of the recessed portion side thereof, and providing a transdermally absorbable drug in one surface of at least the polyether ether ketone resin film among the polyether ether ketone resin film and the adhesive layer to form an intermediate, and subsequently cutting the intermediate into a predetermined size.

According to the first embodiment of the present invention, since the polyether ether ketone resin film is selected rather than using nonwoven fabric or a polyethylene terephthalate resin film as the support layer, there is an effect of being able to improve processability, hydrolysis resistance, chemical resistance, alkaline resistance, and acid resistance of the support layer.

According to the second embodiment, when the polyether ether ketone resin film is used as a sample, and an intradermal reaction test is carried out by using an extract extracted under predetermined conditions in a physiological saline or sesame oil, a difference in average scores of an extract administration site and a blank extract administration site is 1.0 or less in any of extraction media, and thus there is an effect of being able to improve low irritancy of the support layer.

According to the third embodiment, adhesiveness of the adhesive layer can be maintained and contamination of the adhesive layer can be prevented by protecting an adhesive surface of the adhesive layer by using the coating protective layer.

According to the fourth embodiment, a plurality of fine storage cavities can be formed at a low cost. Furthermore, the process steps in forming the storage cavities can be reduced, and simple formation by a simple device can be expected.

According to the fifth embodiment, the intradermal reaction test of the polyether ether ketone resin film is carried out by using an extract extracted in a physiological saline or sesame oil at 121° C. for 1 hour in accordance with “Guidance for Biological Safety Test of Medical Device”, an appendix of Notification No. 20 issued by the Director of Office of Medical Devices Evaluation, Evaluation and Licensing Division, Pharmaceutical and Food Safety Bureau, Pharmaceutical and Medical Safety Bureau, Ministry of Health and Welfare on Mar. 1, 2012 and ISO 10993-10:2010, and thus scores of the intradermal reaction test and a difference in average scores can be specified uniquely by a test method suitable for evaluating safety.

According to the sixth embodiment, the recessed portions for storage cavities are preferably formed by nanoimprinting in one surface of the polyether ether ketone resin film having a crystallization degree of less than 20%, and the crystallization degree of the polyether ether ketone resin film is preferably 20% or more. In such a case, the support layer having a crystallization degree of 20% or more may be formed from the polyether ether ketone resin film having a crystallization degree of less than 20%.

### BRIEF DESCRIPTION OF THE DRAWINGS

FIG. 1 is a cross-sectional explanatory view schematically illustrating an embodiment of a transdermal therapeutic system according to the present invention.

FIG. 2 is a cross-sectional explanatory view schematically illustrating a second embodiment of the transdermal therapeutic system according to the present invention.

### DETAILED DESCRIPTION OF THE INVENTION

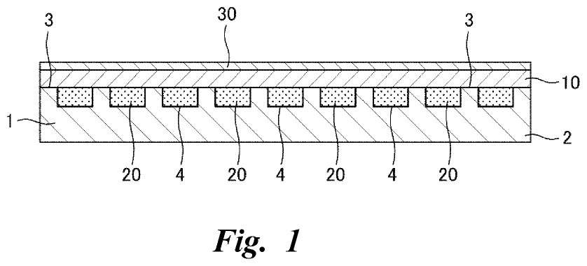

Preferable embodiments of the present invention will be described below with reference to the drawings. As illustrated in FIG. 1, a transdermal therapeutic system (TTS) in the present embodiment is a matrix-type tape-shaped therapeutic system including: a support layer 1 of a flexible tape type; an adhesive layer 10 that is permeable and laminated in the support layer 1; a drug 20 that is transdermally absorbable and provided in at least the support layer 1 among the support layer 1 and the adhesive layer 10; and a liner layer 30 detachably laminated in the adhesive layer 10, and when a polyether ether ketone resin film 2 is selected as the support layer 1, the polyether ether ketone resin film 2 is used as a sample, and an intradermal reaction test is carried out by using an extract extracted under predetermined conditions in a physiological saline or sesame oil, a difference in average scores of an extract administration site and a blank extract administration site is 1.0 or less in any of extraction media.

The support layer 1 is, for example, formed in a planar rectangular shape by using the polyether ether ketone (PEEK) resin film 2 that is a thin film having flexibility, and the adhesive layer 10 is laminated in a surface 3. In addition, the drug 20 is stored in storage cavities 4 that are recessed portions of the surface 3. The polyether ether ketone resin film 2 is formed into a resin film by non-stretching, uniaxial stretching, and biaxially stretching a polyether ether ketone resin that is a thermoplastic resin, and has a thickness set to 1 μm or more and 100 μm or less, preferably 3 μm or more and 50 μm or less, and more preferably 6 μm or more and 30 μm or less to maintain flexibility and not to cause uncomfortable feeling.

The polyether ether ketone resin for the support layer 1 is not particularly limited, but is a crystalline resin having the following repeating units, and has a glass transition temperature of 130° C. or more and 160° C. or less, and a melting point of ordinarily 320° C. or more and 360° C. or less, and is stable and excels in heat resistance up to 500° C. This polyether ether ketone resin is ordinarily used in a form suitable for molding such as a powder form, a grain form, a granular form, and a pellet form.

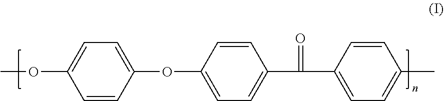

From the perspective of mechanical properties, n of the structural formula of the polyether ether ketone resin is 10 or more, and preferably 20 or more. The polyether ether ketone resin may be a homopolymer including only repeating units of (I), but may have repeating units besides (I). A proportion of the chemical structure of (I) in the polyether ether ketone resin is 50 mol % or more, preferably 70 mol % or more, and more preferably 80 mol % or more with respect to a total of all repeating units constituting the polyether ether ketone resin.

Specific examples of the polyether ether ketone resin include products of Victrex Powder series and Victrex Granule series available from Victrex plc, products of Vestakeep series available from Daicel-Evonik Ltd., and products of KetaSpire polyether ether ketone series available from Solvay Specialty Polymers Japan K.K. Examples of a method for manufacturing the polyether ether ketone resin include methods described in Japanese Patent Application Laid-Open No. Sho 50-27897, Japanese Patent Application Laid-Open No. Sho 51-119797, Japanese Patent Application Laid-Open No. Sho 52-38000, Japanese Patent Application Laid-Open No. Sho 54-90296, Japanese Patent Application No. Sho 55-23574, and Japanese Patent Application No. Sho 56-2091. As the polyether ether ketone resin, within the range that does not impair the effects of the present invention, a block copolymer or random copolymer with another copolymerizable monomer, or a modification thereof can be used.

Physical properties of the polyether ether ketone resin film 2 include, for example, specific gravity of 1.2 or more and 1.4 or less, a tensile elastic modulus of 2000 N/mm² or more and 5000 N/mm² or less, tensile yield strength of 50 N/mm² or more and 100 N/mm² or less, tensile maximum strength of 50 N/mm² or more and 200 N/mm² or less, elongation at tensile breakage of 50% or more and 400% or less, and a water absorption ratio of 1.0 or less. Since there are almost no solvent that can dissolve the polyether ether ketone resin film 2, the polyether ether ketone resin film 2 excels in chemical resistance, hydrolysis resistance, alkaline resistance, acid resistance, and solvent resistance, and can be welded with a laser and printed. Further, since the polyether ether ketone resin film 2 also excels in flame retardancy, and is high in purity, even when combusted, the polyether ether ketone resin film 2 produces no toxic gas.

In addition to the polyether ether ketone resin, a polyimide resin such as a polyimide (PI) resin, a polyamide-imide (PAI) resin, and a polyetherimide (PEI) resin; a polyamide resin such as a polyamide 4T (PA4T) resin, a polyamide 6T (PA6T) resin, a modified polyamide 6T (PA6T) resin, a polyamide 9T (PA9T) resin, a polyamide 10T (PA10T) resin, a polyamide 11T (PA11T) resin, a polyamide 6 (PA6) resin, a polyamide 66 (PA66) resin, and a polyamide 46 (PA46) resin; a polyester resin such as a polyethylene terephthalate (PET) resin, a polybutylene terephthalate (PBT) resin, and a polyethylene naphthalate (PEN) resin; a polyarylene ether ketone resin such as a polyether ketone (PEK) resin, a polyether ether ether (PEEEK) resin, a polyether ketone ketone (PEKK) resin, a polyether ether ketone ketone (PEEKK) resin, and a polyether ketone ether ketone ketone (PEKEKK) resin; a polysulfone resin such as a polysulfone (PSU) resin, a polyether sulfone (PES) resin, and a polyphenylene sulfone (PPSU) resin; a polyarylene sulfide resin such as a polyphenylene sulfide (PPS) resin, a polyphenylene sulfide ketone resin, a polyphenylene sulfide sulfone resin, and a polyphenylene sulfide ketone sulfone resin; a fluororesin such as a polytetrafluoroethylene (PTFE) resin, a polytetrafluoroethylene-perfluoroalkyl vinyl ether copolymer (PFA) resin, a tetrafluoroethylene-hexafluoropropyl copolymer (FEP) resin, a tetrafluoroethylene-ethylene copolymer (ETFE) resin, a polychlorotrifluoroethylene (PCTFE) resin, a polyvinylidene fluoride (PVDF) resin, and a vinylidene fluoride-tetrafluoroethylene-hexafluoropropylene copolymer resin; a liquid crystal polymer (LCP), a polycarbonate (PC) resin, a polyarylate (PAR) resin, and the like can be added as necessary to the polyether ether ketone resin film 2.

Furthermore, in addition to the above-described resins, an antioxidant, a photostabilizer, a colorant, a UV absorber, a plasticizer, an antistatic agent, a heat resistance enhancer, a flame retardant, an inorganic compound, an organic compound, a resin modifier, and the like can be added selectively to the polyether ether ketone resin film 2 within the range that does not impair the properties of the present invention.

The polyether ether ketone resin film 2 is subjected to an intradermal reaction test from the perspective of ensuring low irritancy to skin. In such a case, the polyether ether ketone resin film 2 is used as a sample, and the test is carried out by using an extract extracted in a physiological saline or sesame oil at 121° C. for 1 hour, in accordance with “Guidance for Biological Safety Test of Medical Device”, an appendix of Notification No. 20 issued by the Director of Office of Medical Devices Evaluation, Evaluation and Licensing Division, Pharmaceutical and Food Safety Bureau, Pharmaceutical and Medical Safety Bureau, Ministry of Health and Welfare on Mar. 1, 2012 regarding the “Basic Principles of Biological Safety Evaluation Required for Application for Approval to Market Medical Devices,” that is suitable for risk assessment, and in accordance with ISO 10993-10:2010, Biological evaluation of medical devices—Part 10: Tests for irritation and skin sensitization and numerical values relating to low irritancy are uniquely specified.

As a result of such an intradermal reaction test, when a difference in average scores of an extract administration site and a blank extract administration site is 1.0 or less in any of extraction media, it is determined that the polyether ether ketone resin film 2 has low irritancy to skin and can be used. On the other hand, when the difference in average scores exceeds 1.0, it is determined that there is a problem in irritancy to skin, and the polyether ether ketone resin film 2 is not adopted.

In a planar direction of the surface 3 of the polyether ether ketone resin film 2 that is laminated in the adhesive layer 10, a plurality of storage cavities 4 for a drug are formed as a plurality of recesses by a nanoimprinting method, and each of the storage cavities 4 is formed in a minute recessed portion of a micrometer scale, and each storage cavity 4 that is fine is filled with and stores the drug 20. The nanoimprinting method is a method including pressing a mold against the polyether ether ketone resin film 2 and transferring a fine pattern of the mold on a nanometer order to the polyether ether ketone resin film 2. According to the nanoimprinting method, an array pattern of the plurality of storage cavities 4 that are minute can be formed at a low cost, and moreover, the number of process steps is small, and simple formation can be expected with a simple device.

The surface 3 of the polyether ether ketone resin film 2 in which the adhesive layer 10 is laminated is preferably subjected to a surface treatment such as, for example, a corona discharge treatment, a plasma treatment, an oxidation treatment, a ultraviolet treatment, a flame treatment, an ITRO treatment, a hairline finishing, and sand-mat finishing to improve cleaning, adhesiveness and the like.

The adhesive layer 10 is laminated and formed by coating the surface 3 of the polyether ether ketone resin film 2 with a predetermined adhesive and drying, and is caused to adhere in close contact to skin. The predetermined adhesive agent is not particularly limited, but examples thereof include an acrylic adhesive that is stable with respect to light and oxygen and that does not easily cause development of a rash, a rubber adhesive excelling in uniform quality, and a silicone adhesive excelling in thermal aging resistance, chemical resistance, water resistance, releasability of the drug 20 and the like. One type of adhesive can be used alone, or a combination of two or more adhesives can be used. Furthermore, from the perspective of ensuring flexibility and the like, a thickness of the adhesive layer 10 is 1 μm or more and 100 μm or less, preferably 10 μm or more and 80 μm or less, and more preferably 20 μm or more and 60 μm or less.

The drug 20 is not limited, but includes, for example, scopolamine, nitroglycerin, isosorbide nitrate, clonidine, tulobuterol, estradiol, fentanyl or the like that excels in transdermal absorbability. The plurality of storage cavities 4 of the polyether ether ketone resin film 2 are coated and filled with a small effective amount of the drug 20 for prevention or treatment a disease of interest, and are dried to storage the drug 20.

The liner layer 30 is not limited, but may include, for example, a planar rectangular polyether ether ketone resin film, a polyolefinic resin film such as a polypropylene resin film, and a polyethylene resin film; a polyester resin film such as a polyethylene terephthalate resin film, a polybutylene terephthalate resin film, and a polyethylene naphthalate resin film; a polyamide resin film such as a polyamide 6 resin film, a polyamide 66 resin film, and a polyamide 9T resin film; a polyphenylene sulfide resin film, or a release paper. These resin films can be used as a resin film having any shape such as an unstretched film, a uniaxially stretched film, and a biaxially stretched film. Such a liner layer 30 functions to cover and protect an adhesive surface that is a surface of the adhesive layer 10.

In the above-described configuration, when the transdermal therapeutic system is manufactured, the polyether ether ketone resin film 2 serving as the support layer 1 is first formed with a polyether ether ketone resin. At this time, a crystallization degree of the polyether ether ketone resin film 2 is preferably less than 20%. This is because when the crystallization degree of the polyether ether ketone resin film 2 is 20% or more, softening of the polyether ether ketone resin film 2 is impaired, and a problem occurs in thermoformability such as nanoimprint molding of the polyether ether ketone resin film 2.

The crystallization degree of the polyether ether ketone resin film 2 is calculated by the following equation based on thermal analysis results using a differential scanning calorimeter.

Crystallization Degree (%)={(ΔHm−ΔHc)/ΔHx}×100 (Eq.1)

Here, ΔHm: Calorific value (J/g) of a crystal melting peak of the polyether ether ketone resin film 2

ΔHc: Calorific value (J/g) of a recrystallization peak of the polyether ether ketone resin film 2

ΔHx: Theoretical value of melting energy of the polyether ether ketone resin film 2 that is 100% crystallized, and the theoretical value is 130 J/g.

The polyether ether ketone resin film 2 can be manufactured by a known manufacturing method such as melt extrusion molding, calender molding, and casting, but from the perspectives of handling ease and equipment simplification, the polyether ether ketone resin film 2 is preferably continuously and thinly extruded and formed by melt extrusion molding. Here, the melt extrusion molding refers to a method in which a polyether ether ketone resin is introduced into a melt extrusion molding machine and melt-kneaded, and the polyether ether ketone resin film 2 having a band shape is continuously extruded from a T-die of the melt extrusion molding machine.

From the perspective of thermoformability, optimally, a crystallization degree of the polyether ether ketone resin film 2 extruded and formed is less than 20% as described above, but is preferably less than 15%, and more preferably less than 10%. On the other hand, a lower limit of the crystallization degree of the polyether ether ketone resin film 2 is not particularly limited, but is preferably 1% or more.

When the polyether ether ketone resin film 2 is extruded and formed, the polyether ether ketone resin film 2 is sequentially wound on a plurality of pressure rolls, cooling rolls, tension rolls, and winding tubes of a winder, and after the polyether ether ketone resin film 2 is cooled by the cooling rolls, both end portions of the polyether ether ketone resin film 2 are cut with a slitting blade, and sequentially wound on a winding tube to manufacture the polyether ether ketone resin film 2.

Here, the plurality of pressure rolls and cooling rolls are adjusted to have a temperature of glass transition temperature; Tg of the polyether ether ketone resin film 2+20° C. or less, preferably a glass transition temperature; Tg of the polyether ether ketone resin film 2 or less, and more preferably 50° C. or more and glass transition temperature; Tg of the polyether ether ketone resin film 2−10° C. or less. This is because when a temperature of the plurality of pressure bonding rolls and cooling rolls exceeds glass transition temperature; Tg of the polyether ether ketone resin film 2+20° C., crystallization of the polyether ether ketone resin film 2 advances, the crystallization degree of the polyether ether ketone resin film 2 becomes 20% or more, and the polyether ether ketone resin film 2 does not soften, and thus thermoformability by the subsequent nanoimprinting method deteriorates.

A portion of the polyether ether ketone resin film 2 manufactured is then cut and used as a sample, and the above-described intradermal reaction test is carried out to examine low irritancy of the polyether ether ketone resin film 2. As a result of the intradermal reaction test, when a difference in average scores of an extract administration site and a blank extract administration site is 1.0 or less in any of extraction media, it is determined that the polyether ether ketone resin film 2 manufactured has low irritancy, and the polyether ether ketone resin film 2 manufactured is used as a good quality product.

Next, the plurality of storage cavities 4 that are minute are formed as recesses in the surface 3 of the polyether ether ketone resin film 2 by a nanoimprinting method. To soften the polyether ether ketone resin film 2 when the plurality of storage cavities 4 that are minute are formed as recesses in the surface 3 of the polyether ether ketone resin film 2 by a nanoimprinting method, the polyether ether ketone resin film 2 is preheated in the temperature range of glass transition temperature; Tg of the polyether ether ketone resin film 2−10° C. or more and [glass transition temperature; Tg of the polyether ether ketone resin film 2+50° C. or less, preferably in the temperature range of the glass transition temperature; Tg of the polyether ether ketone resin film 2 or more and glass transition temperature; Tg of the polyether ether ketone resin film 2+30° C. or less, and more preferably in the temperature range of the glass transition temperature; Tg of the polyether ether ketone resin film 2 or more and glass transition temperature; Tg of the polyether ether ketone resin film 2+20° C., and subsequently, from the perspective of preventing deformation and the like of the polyether ether ketone resin film 2, the storage cavities 4 are formed as recesses in the temperature range of the glass transition temperature; Tg of the polyether ether ketone resin film 2 or more and less than a melting point of the polyether ether ketone resin film 2, preferably in the temperature range of glass transition temperature; Tg of the polyether ether ketone resin film 2+30° C. or more and melting point of the polyether ether ketone resin film 2−50° C. or less, and more preferably in the temperature range of glass transition temperature; Tg of the polyether ether ketone resin film 2+30° C. or more and melting point of the polyether ether ketone resin film 2−100° C. or less.

Optimally, a crystallization degree of the polyether ether ketone resin film 2 in which the plurality of storage cavities 4 are nano-imprinted and formed is 20% or more, preferably 23% or more, more preferably 25% or more, and even more preferably 27% or more to improve hydrolysis resistance, chemical resistance, alkaline resistance, acid resistance, and low irritancy, and contribute to physical stability of the storage cavities 4. On the other hand, an upper limit of the crystallization degree of the polyether ether ketone resin film 2 is not particularly limited, but 50% or less is preferable. This is because when the crystallization degree of the polyether ether ketone resin film 2 exceeds 50%, heat treatment for a long period of time is required, and a problem occurs in productivity.

Examples of a method for improving the crystallization degree of the polyether ether ketone resin film 2 in which the plurality of storage cavities 4 are nano-imprinted and formed include a method that is performed simultaneously with the formation of recesses by a nanoimprinting method, and a method according to a heat treatment method or the like that is performed after the formation of recesses by a nanoimprinting method. The heat treatment performed after the formation of recesses by a nanoimprinting method is performed in the temperature range of the glass transition temperature; Tg of the polyether ether ketone resin film 2 or more and less than the melting point of the polyether ether ketone resin film 2, and preferably in the temperature range of glass transition temperature; Tg of the polyether ether ketone resin film 2+30° C. or more and melting point of the polyether ether ketone resin film 2−50° C. or less. However, from the perspective of equipment simplification, improvement in the crystallization degree of the polyether ether ketone resin film 2 may be made simultaneously with the formation of recesses of the storage cavities 4 by a nanoimprinting method.

After the plurality of storage cavities 4 are formed as recesses in the surface 3 of the polyether ether ketone resin film 2 by nanoimprint molding, the plurality of storage cavities 4 in the polyether ether ketone resin film 2 manufactured are each coated and filled with the drug 20 that is transdermally absorbable, and the drug 20 is dried and stored in each of the storage cavities 4.

Next, the adhesive layer 10 is laminated and formed by thinly coating the surface 3 of the polyether ether ketone resin film 2 with an adhesive and drying the adhesive, and the liner layer 30 is laminated and bonded to the adhesive surface of the adhesive layer 10 to form an intermediate. After the intermediate is formed, a transdermal therapeutic system can be manufactured by cutting the intermediate into a predetermined size to insert an incision in the liner layer 30 thereof, and cutting the intermediate into the size of the polyether ether ketone resin film 2, and packaging and inspecting.

Next, when the transdermal therapeutic system is used, the liner layer 30 may be peeled from the adhesive layer 10, and the adhesive layer 10 may be caused to adhere to skin. Then, the drug 20 permeates the adhesive layer 10 from the storage cavities 4 of the polyether ether ketone resin film 2 and is distributed to a skin surface, and diffuses into skin to transfer subcutaneously.

According to the above, since the polyether ether ketone resin film 2 is used as the support layer 1, the support layer 1 that exhibits more excellent processability, hydrolysis resistance, chemical resistance, acid resistance, and alkaline resistance than those of a conventional support layer can be obtained. Furthermore, mechanical properties, heat resistance, solvent resistance, wear resistance, slidability, dimensional stability, flame retardancy, and the like of the support layer 1 can be improved significantly by selecting the polyether ether ketone resin film 2. Furthermore, since the polyether ether ketone resin film 2 having a difference in average scores of 1.0 or less in the specified intradermal reaction test is selected and used rather than simply using the polyether ether ketone resin film 2, low irritancy to skin of the polyether ether ketone resin film 2 can be ensured, and improvement in stability with respect to at least a physiological saline, sesame oil or the like can be expected.

Furthermore, since the plurality of storage cavities 4 are formed by a nanoimprinting method, excellent dimensional control enables high precision control of release of the drug 20. Further, since the transdermal therapeutic system is a matrix-type transdermal therapeutic system, a structure of the therapeutic system is simple, a potential for release of an excessive amount of the drug 20 is low, a dosage can be adjusted easily by area, and conformability to skin can be improved.

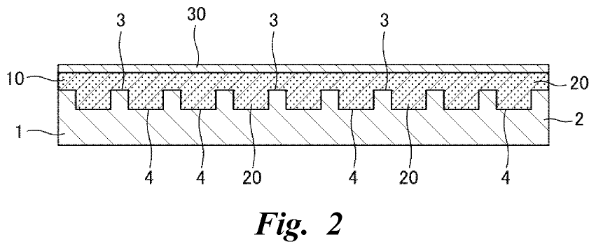

Next, FIG. 2 illustrates a second embodiment of the present invention, and in this case, an adhesive layer 10 and a drug 20 are integrated by blending an adhesive of the adhesive layer 10 and the drug 20 at the time of manufacturing a transdermal therapeutic system, and the drug 20 is also stored in the adhesive layer 10.

In the present embodiment, when the transdermal therapeutic system is manufactured, a blended composition may be prepared by stirring and blending the adhesive of the adhesive layer 10 and the drug 20, and a surface 3 of a polyether ether ketone resin film 2 having good quality may be coated thickly with the blended composition. In addition, each of a plurality of storage cavities 4 of the polyether ether ketone resin film 2 may be filled with the blended composition, and after the blended composition is dried to laminate and form the adhesive layer 10 containing the drug 20, a liner layer 30 may be laminated detachably in an adhesive surface of the adhesive layer 10 to form an intermediate. The other portions are the same as those of the embodiment described above, and thus descriptions thereof will be omitted.

The same action and effects as those of the above-described embodiment can also be expected in the present embodiment, and moreover, since it is not necessary to laminate and form the adhesive layer 10 by coating and drying of the adhesive after coating, filling and drying of the drug 20, it is clear that manufacturing work can be simplified and accelerated. Furthermore, since the adhesive layer 10 and the drug 20 are integrated, and the drug 20 is also stored in the adhesive layer 10, a significant increase in a usage amount of the drug 20 can be expected.

Note that in the embodiment described above, the polyether ether ketone resin film 2 having a planar rectangular shape is described, but the polyether ether ketone resin film 2 having a planar circle, an elliptical shape or the like may be used. Furthermore, in the embodiment described above, the adhesive layer 10 is laminated and formed by coating and drying of the adhesive, but the adhesive layer 10 is not limited in any way thereto.

For example, after the plurality of storage cavities 4 of the polyether ether ketone resin film 2 are coated and filled with the drug 20 and the drug 20 is dried, a resin film may be laminated in the surface 3 of the polyether ether ketone resin film 2, and a surface of this resin film may be coated with an adhesive and the adhesive is dried to laminate and form the adhesive layer 10. This resin film may be perforated to include multiple cavities, and these multiple cavities may be used as drug release control cavities to constantly maintain a permeation rate of the drug 20 for a long period of time.

### Examples

Examples of transdermal therapeutic systems according to the present invention will be described below.

A polyether ether ketone resin film (available from Shin-Etsu Polymer Co., Ltd.: product name Shin-Etsu Sepla Film PEEK (trade name)) was prepared, and the polyetherether ketone resin film was subjected to an intradermal reaction test.

In this intradermal reaction test, the polyether ether ketone resin film was used as a sample, and the test was carried out by using an extract extracted in a physiological saline or sesame oil at 121° C. for 1 hour in accordance with Notification No. 20 issued by the Director of Office of Medical Devices Evaluation, Evaluation and Licensing Division, Pharmaceutical and Food Safety Bureau, Pharmaceutical and Medical Safety Bureau, Ministry of Health and Welfare on Mar. 1, 2012 regarding the “Basic Principles of Biological Safety Evaluation Required for Application for Approval to Market Medical Devices” that is suitable for risk assessment, and in accordance with ISO 10993-10:2010, “Biological evaluation of medical devices—Part 10: Tests for irritation and skin sensitization”.

### [Preparation of Extract]

### 1) Physiological Saline Extraction

First, a sample was collected from the polyether ether ketone resin film and cut into a size of approximately 1.5 cm×1.5 cm, a physiological saline (Pharmacopoeia) was added at a ratio of 1 mL with respect to 6 cm² of surface area as one surface of the sample, and an extract was extracted at 121° C. for 1 hour. After the extraction, the extract was left to cool at room temperature, and vigorously shaken, but since suspended matter was observed, a supernatant liquid obtained by centrifugation for 5 minutes at 3600 rpm was used as an extract.

Furthermore, for the physiological saline, a blank extract was separately prepared in the same method as the extract. When pH levels of the extract and the blank extract were confirmed, the pH levels of both the extract and the blank extract were confirmed to be 5. The extract and the blank extract were stored at room temperature, and were used in the intradermal reaction test within 24 hours after the extraction treatment ended.

### 2) Sesame Oil Extraction

First, a sample was collected from the polyether ether ketone resin film and cut into a size of approximately 1.5 cm×1.5 cm, sesame oil (Pharmacopoeia) was added at a ratio of 1 mL with respect to 6 cm² of surface area (one surface) of the sample, and an extract was extracted at 121° C. for 1 hour. After the extraction, the extract was left to cool at room temperature and was used as an extract.

Furthermore, for the sesame oil, a blank extract was separately prepared in the same method as the extract. The extract and the blank extract were stored at room temperature, and were used in the intradermal reaction test within 24 hours after the extraction treatment ended.

### [Test Animals and Breeding Conditions]

### 1) Test Animals

Species/Lineage/Sex: rabbit/Japanese white rabbit/male

Microbial Control: healthy

Supply Source: Kitayama Labes Co., Ltd.

Selection of animals used: Animals that met the following conditions were used for the intradermal reaction test.

a) Animals that were preliminarily raised for one week or more and confirmed to have no abnormalities in general conditions

b) Animals with a body weight of 2 kg or more on the day of administration

### 2) Breeding Conditions

Temperature: 20 to 26° C.

Relative Humidity: 30 to 80%

Illumination Time: 12 hours (lighting turned on at 8 AM, and turned off at 8 PM)

Cage: Individual containment in FRP cages

Feed: Restricted supply of gamma irradiated feed for rabbits and guinea pigs, LRC4, available from Oriental Yeast Co., Ltd.

Drinking water: Automatic intake of tap water through automatic supply of water

### [Test Method]

Electric clippers were used to shear a dorsal coat of hair of each of three test animals, and with the spinal column gripped from both sides, each of 0.2 mL of the physiological saline extract and 0.2 mL of the blank extract was administered intradermally in five places on one side, and each of 0.2 mL of the sesame oil extract and 0.2 mL of the blank extract was administered intradermally in five places on the opposite side.

The administration sites were observed immediately after the administration, and at 24 hours, 48 hours, and 72 hours after the administration, and skin reaction was scored according to criteria shown in Table 1. In scoring, scores for erythema and edema at each of the extract administration sites and the blank extract administration d sites of the three test animals were totaled, and an average of the scores was calculated by using equation 1 for each extraction medium. The average score of the blank extract administration site was subtracted from the average score of the extract administration site, and when the obtained value was 1.0 or less, irritancy of the extract was evaluated as being “absent or negligible.” Otherwise, the extract was evaluated to have irritancy.

Note that the observation results immediately after the administration were not used in calculating the average score because the effect of the administration still remained.

Furthermore, when a difference in the average scores was 0.0 or less, a value of 0 was used. The weight of each test animal was measured on the day of administration and on the day of the end of observation.

Average score=(total score for erythema and edema at 24 hours, 48 hours, and 72 hours for all test animals)/45 (Eq. 2)

Here, a constant 45 is a product of 3 for the number of the test animals, 3 for the number of observation points; 24 hours, 48 hours, and 72 hours after the administration, and 5 for the number of the administration sites.

TABLE 1
Skin (Intradermal) Reaction Scoring System

| | |
|---|---|
| Formation of erythema and scab | |
| No erythema | 0 |
| Very mild erythema (barely recognizable) | 1 |
| Clear erythema | 2 |
| Moderate or advanced erythema | 3 |
| Slight scab formation (up to deep damage) | 4 |
| from advanced erythema | |
| | [Maximum |
| | Score: 4] |
| Formation of edema | |
| No edema | 0 |
| Very mild edema (barely recognizable) | 1 |
| Mild edema (Distinct edge can be identified by | 2 |
| sharp bulging.) | |
| Moderate edema (Bulging of about 1 mm) | 3 |
| Advanced edema (Bulging of 1 mm or more and | 4 |
| spreading beyond an exposure range) | |
| | [Maximum |
| | Score: 4] |
| | [Maximum score |
| | of total score of |
| | erythema/scab |
| | and edema: 8] |

### [Test Results]

### 1) Weight Change

The weights of the test animals on the day of administration and on the day of the end of observation are shown in Table 2.

TABLE 2
Weights of Test Animals

| | Weight | |
|---|---|---|
| Animal No. | Day of Administration | Day of End of Observation |
| 1 | 3.04 | 3.04 |
| 2 | 3.19 | 3.17 |
| 3 | 3.32 | 3.21 |

Unit: kg

### 2) Physiological Saline Extraction

Skin reaction was not observed in any of the extract administration site and the blank extract administration site. Furthermore, the average scores at 24 hours, 48 hours, and 72 hours after the administration were calculated as 0/45 in both the extract administration site and the blank extract administration site, and a difference in the average scores was 0 (see Table 3 and Table 4).

TABLE 3
Score Results at Each Observation Time (Extraction
medium: physiological saline)

| Observation time after administration | | | |
|---|---|---|---|
| Immediately | | | |
| after | 24 h | 48 h | 72 h |

Animal No. 1

| | | | | | | |
|---|---|---|---|---|---|---|
| Administration | Extract | 1 | 0/— | 0/0 | 0/0 | 0/0 |
| site | | 2 | 0/— | 0/0 | 0/0 | 0/0 |
| | | 3 | 0/— | 0/0 | 0/0 | 0/0 |
| | | 4 | 0/— | 0/0 | 0/0 | 0/0 |
| | | 5 | 0/— | 0/0 | 0/0 | 0/0 |
| | Blank | 6 | 0/— | 0/0 | 0/0 | 0/0 |
| | extract | 7 | 0/— | 0/0 | 0/0 | 0/0 |
| | | 8 | 0/— | 0/0 | 0/0 | 0/0 |
| | | 9 | 0/— | 0/0 | 0/0 | 0/0 |
| | | 10 | 0/— | 0/0 | 0/0 | 0/0 |

Animal No. 2

| | | | | | | |
|---|---|---|---|---|---|---|
| Administration | Extract | 1 | 0/— | 0/0 | 0/0 | 0/0 |
| site | | 2 | 0/— | 0/0 | 0/0 | 0/0 |
| | | 3 | 0/— | 0/0 | 0/0 | 0/0 |
| | | 4 | 0/— | 0/0 | 0/0 | 0/0 |
| | | 5 | 0/— | 0/0 | 0/0 | 0/0 |
| | Blank | 6 | 0/— | 0/0 | 0/0 | 0/0 |
| | extract | 7 | 0/— | 0/0 | 0/0 | 0/0 |
| | | 8 | 0/— | 0/0 | 0/0 | 0/0 |
| | | 9 | 0/— | 0/0 | 0/0 | 0/0 |
| | | 10 | 0/— | 0/0 | 0/0 | 0/0 |

Animal No. 3

| | | | | | | |
|---|---|---|---|---|---|---|
| Administration | Extract | 1 | 0/— | 0/0 | 0/0 | 0/0 |
| site | | 2 | 0/— | 0/0 | 0/0 | 0/0 |
| | | 3 | 0/— | 0/0 | 0/0 | 0/0 |
| | | 4 | 0/— | 0/0 | 0/0 | 0/0 |
| | | 5 | 0/— | 0/0 | 0/0 | 0/0 |
| | Blank | 6 | 0/— | 0/0 | 0/0 | 0/0 |
| | extract | 7 | 0/— | 0/0 | 0/0 | 0/0 |
| | | 8 | 0/— | 0/0 | 0/0 | 0/0 |
| | | 9 | 0/— | 0/0 | 0/0 | 0/0 |
| | | 10 | 0/— | 0/0 | 0/0 | 0/0 |

The results are shown in the order of erythema and scab/edema.
—: Was not scored because the effect of the administration remained.

TABLE 4
Average Score for Skin Reaction (Extraction
media: physiological saline)

| Admin- | | Total of Scores | | | | | | |
|---|---|---|---|---|---|---|---|---|
| istration | Animal | Observation time | | | Sub- | | Average | Differ- |
| Material | No. | 24 h | 48 h | 72 h | Total | Total | Score | ence |
| Extract | 1 | 0 | 0 | 0 | 0 | 0 | 0/45 | 0 |
| | 2 | 0 | 0 | 0 | 0 | | | |
| | 3 | 0 | 0 | 0 | 0 | | | |
| Blank | 1 | 0 | 0 | 0 | 0 | 0 | 0/45 | |
| extract | 2 | 0 | 0 | 0 | 0 | | | |
| | 3 | 0 | 0 | 0 | 0 | | | |

### 3) Sesame Oil Extraction

Very mild erythema (score of 1) was confirmed in any of the extract administration site and the blank extract administration site. Furthermore, the average scores at 24 hours, 48 hours, and 72 hours after the administration were calculated as 45/45 in both the extract administration site and the blank extract administration site, and a difference in the average scores was 0 (see Table 5 and Table 6).

TABLE 5
Score Results at Each Observation Time
(Extraction medium: sesame oil)

| Observation time after administration | | | |
|---|---|---|---|
| Immediately | | | |
| after | 24 h | 48 h | 72 h |

Animal No. 1

| | | | | | | |
|---|---|---|---|---|---|---|
| Administration | Extract | 11 | 1/— | 1/0 | 1/0 | 1/0 |
| site | | 12 | 1/— | 1/0 | 1/0 | 1/0 |
| | | 13 | 1/— | 1/0 | 1/0 | 1/0 |
| | | 14 | 1/— | 1/0 | 1/0 | 1/0 |
| | | 15 | 1/— | 1/0 | 1/0 | 1/0 |
| | Blank | 16 | 1/— | 1/0 | 1/0 | 1/0 |
| | extract | 17 | 1/— | 1/0 | 1/0 | 1/0 |
| | | 18 | 1/— | 1/0 | 1/0 | 1/0 |
| | | 19 | 1/— | 1/0 | 1/0 | 1/0 |
| | | 20 | 1/— | 1/0 | 1/0 | 1/0 |

Animal No. 2

| | | | | | | |
|---|---|---|---|---|---|---|
| Administration | Extract | 11 | 1/— | 1/0 | 1/0 | 1/0 |
| site | | 12 | 1/— | 1/0 | 1/0 | 1/0 |
| | | 13 | 1/— | 1/0 | 1/0 | 1/0 |
| | | 14 | 1/— | 1/0 | 1/0 | 1/0 |
| | | 15 | 1/— | 1/0 | 1/0 | 1/0 |
| | Blank | 16 | 1/— | 1/0 | 1/0 | 1/0 |
| | extract | 17 | 1/— | 1/0 | 1/0 | 1/0 |
| | | 18 | 1/— | 1/0 | 1/0 | 1/0 |
| | | 19 | 1/— | 1/0 | 1/0 | 1/0 |
| | | 20 | 1/— | 1/0 | 1/0 | 1/0 |

Animal No. 3

| | | | | | | |
|---|---|---|---|---|---|---|
| Administration | Extract | 11 | 1/— | 1/0 | 1/0 | 1/0 |
| site | | 12 | 1/— | 1/0 | 1/0 | 1/0 |
| | | 13 | 1/— | 1/0 | 1/0 | 1/0 |
| | | 14 | 1/— | 1/0 | 1/0 | 1/0 |
| | | 15 | 1/— | 1/0 | 1/0 | 1/0 |
| | Blank | 16 | 1/— | 1/0 | 1/0 | 1/0 |
| | extract | 17 | 1/— | 1/0 | 1/0 | 1/0 |
| | | 18 | 1/— | 1/0 | 1/0 | 1/0 |
| | | 19 | 1/— | 1/0 | 1/0 | 1/0 |
| | | 20 | 1/— | 1/0 | 1/0 | 1/0 |

The results are shown in the order of erythema and scab/edema.
—: Was not scored because the effect of the administration remained.

TABLE 6
Average Score for Skin Reaction (Extraction media: sesame oil)

| Admin- | | Total of Scores | | | | | | |
|---|---|---|---|---|---|---|---|---|
| istration | Animal | Observation time | | | Sub- | | Average | Differ- |
| Material | No. | 24 h | 48 h | 72 h | Total | Total | Score | ence |
| Extract | 1 | 5 | 5 | 5 | 15 | 45 | 45/45 | 0 |
| | 2 | 5 | 5 | 5 | 15 | | | |
| | 3 | 5 | 5 | 5 | 15 | | | |
| Blank | 1 | 5 | 5 | 5 | 15 | 45 | 45/45 | |
| extract | 2 | 5 | 5 | 5 | 15 | | | |
| | 3 | 5 | 5 | 5 | 15 | | | |

### CONCLUSION

In any of the extraction media, a difference in the average scores of the extract and the blank extract was 1.0 or less. From this result, the polyether ether ketone resin film was able to have irritancy of the saline solution extract and the sesame oil extract evaluated as being “absent or negligible,” and was confirmed to be appropriate as a support layer of a transdermal therapeutic system.

1. A transdermal therapeutic system in which an adhesive layer is laminated in a support layer, and a transdermally absorbable drug is provided in at least the support layer among the support layer and the adhesive layer, wherein the support layer is a polyether ether ketone resin film.

2. The transdermal therapeutic system as described in claim 1, wherein when the polyether ether ketone resin film is used as a sample, and an intradermal reaction test is carried out by using an extract extracted under predetermined conditions in a physiological saline or sesame oil, a difference in average scores of an extract administration site and a blank extract administration site is 1.0 or less in any of extraction media.

3. The transdermal therapeutic system as described in claim 1, comprising a coating protective layer that is detachably laminated in the adhesive layer.

4. The transdermal therapeutic system as described in claim 2, comprising a coating protective layer that is detachably laminated in the adhesive layer.

5. The transdermal therapeutic system as described in claim 1, wherein a plurality of storage cavities for a drug are formed by a nanoimprinting method in a laminated surface that is laminated in the adhesive layer of the polyether ether ketone resin film.

6. The transdermal therapeutic system as described in claim 2, wherein a plurality of storage cavities for a drug are formed by a nanoimprinting method in a laminated surface that is laminated in the adhesive layer of the polyether ether ketone resin film.

7. The transdermal therapeutic system as described in claim 3, wherein a plurality of storage cavities for a drug are formed by a nanoimprinting method in a laminated surface that is laminated in the adhesive layer of the polyether ether ketone resin film.

8. The transdermal therapeutic system as described in claim 4, wherein a plurality of storage cavities for a drug are formed by a nanoimprinting method in a laminated surface that is laminated in the adhesive layer of the polyether ether ketone resin film.

9. The transdermal therapeutic system according to claim 1, wherein the intradermal reaction test of the polyether ether ketone resin film is carried out by using an extract extracted in a physiological saline or sesame oil at 121° C. for 1 hour in accordance with “Guidance for Biological Safety Test of Medical Device”, an appendix of Notification No. 20 issued by the Director of Office of Medical Devices Evaluation, Evaluation and Licensing Division, Pharmaceutical and Food Safety Bureau, Pharmaceutical and Medical Safety Bureau, Ministry of Health and Welfare on Mar. 1, 2012 and ISO 10993-10:2010.

10. The transdermal therapeutic system according to claim 2, wherein the intradermal reaction test of the polyether ether ketone resin film is carried out by using an extract extracted in a physiological saline or sesame oil at 121° C. for 1 hour in accordance with “Guidance for Biological Safety Test of Medical Device”, an appendix of Notification No. 20 issued by the Director of Office of Medical Devices Evaluation, Evaluation and Licensing Division, Pharmaceutical and Food Safety Bureau, Pharmaceutical and Medical Safety Bureau, Ministry of Health and Welfare on Mar. 1, 2012 and ISO 10993-10:2010.

11. The transdermal therapeutic system according to claim 3, wherein the intradermal reaction test of the polyether ether ketone resin film is carried out by using an extract extracted in a physiological saline or sesame oil at 121° C. for 1 hour in accordance with “Guidance for Biological Safety Test of Medical Device”, an appendix of Notification No. 20 issued by the Director of Office of Medical Devices Evaluation, Evaluation and Licensing Division, Pharmaceutical and Food Safety Bureau, Pharmaceutical and Medical Safety Bureau, Ministry of Health and Welfare on Mar. 1, 2012 and ISO 10993-10:2010.

12. The transdermal therapeutic system according to claim 4, wherein the intradermal reaction test of the polyether ether ketone resin film is carried out by using an extract extracted in a physiological saline or sesame oil at 121° C. for 1 hour in accordance with “Guidance for Biological Safety Test of Medical Device”, an appendix of Notification No. 20 issued by the Director of Office of Medical Devices Evaluation, Evaluation and Licensing Division, Pharmaceutical and Food Safety Bureau, Pharmaceutical and Medical Safety Bureau, Ministry of Health and Welfare on Mar. 1, 2012 and ISO 10993-10:2010.

13. The transdermal therapeutic system according to claim 5, wherein the intradermal reaction test of the polyether ether ketone resin film is carried out by using an extract extracted in a physiological saline or sesame oil at 121° C. for 1 hour in accordance with “Guidance for Biological Safety Test of Medical Device”, an appendix of Notification No. 20 issued by the Director of Office of Medical Devices Evaluation, Evaluation and Licensing Division, Pharmaceutical and Food Safety Bureau, Pharmaceutical and Medical Safety Bureau, Ministry of Health and Welfare on Mar. 1, 2012 and ISO 10993-10:2010.

14. The transdermal therapeutic system according to claim 6, wherein the intradermal reaction test of the polyether ether ketone resin film is carried out by using an extract extracted in a physiological saline or sesame oil at 121° C. for 1 hour in accordance with “Guidance for Biological Safety Test of Medical Device”, an appendix of Notification No. 20 issued by the Director of Office of Medical Devices Evaluation, Evaluation and Licensing Division, Pharmaceutical and Food Safety Bureau, Pharmaceutical and Medical Safety Bureau, Ministry of Health and Welfare on Mar. 1, 2012 and ISO 10993-10:2010.

15. The transdermal therapeutic system according to claim 7, wherein the intradermal reaction test of the polyether ether ketone resin film is carried out by using an extract extracted in a physiological saline or sesame oil at 121° C. for 1 hour in accordance with “Guidance for Biological Safety Test of Medical Device”, an appendix of Notification No. 20 issued by the Director of Office of Medical Devices Evaluation, Evaluation and Licensing Division, Pharmaceutical and Food Safety Bureau, Pharmaceutical and Medical Safety Bureau, Ministry of Health and Welfare on Mar. 1, 2012 and ISO 10993-10:2010.

16. The transdermal therapeutic system according to claim 8, wherein the intradermal reaction test of the polyether ether ketone resin film is carried out by using an extract extracted in a physiological saline or sesame oil at 121° C. for 1 hour in accordance with “Guidance for Biological Safety Test of Medical Device”, an appendix of Notification No. 20 issued by the Director of Office of Medical Devices Evaluation, Evaluation and Licensing Division, Pharmaceutical and Food Safety Bureau, Pharmaceutical and Medical Safety Bureau, Ministry of Health and Welfare on Mar. 1, 2012 and ISO 10993-10:2010.

17. The transdermal therapeutic system of claim 5, wherein a crystallization degree of the polyether ether ketone resin film is less than 20%.

18. The transdermal therapeutic system of claim 6, wherein a crystallization degree of the polyether ether ketone resin film is less than 20%.

19. The transdermal therapeutic system of claim 7, wherein a crystallization degree of the polyether ether ketone resin film is less than 20%.

20. The transdermal therapeutic system of claim 8, wherein a crystallization degree of the polyether ether ketone resin film is less than 20%.
